(19) United States
(12) Patent Application Publication
(10) Pub. No.: US 2026/0369370 A1
(43) Pub. Date: Nov. 19, 2026

(54) ELECTRONIC MEDICAL RECORD GENERATION METHODS, TASK EXECUTION METHODS, DEVICES, MEDIA, AND PRODUCTS

(71) Applicant: Alipay (Hangzhou) Digital Service Technology Co., Ltd., Hangzhou (CN)

(72) Inventors: Jie FENG, Hangzhou (CN); Dan YANG, Hangzhou (CN); Chengyuan ZHAO, Hangzhou (CN); Yue SHEN, Hangzhou (CN); He SHENG, Hangzhou (CN); Jian WANG, Hangzhou (CN); Peng WEI, Hangzhou (CN)

(73) Assignee: Alipay (Hangzhou) Digital Service Technology Co., Ltd., Hangzhou (CN)

(21) Appl. No.: 19/681,602

(22) Filed: May 19, 2026

(30) Foreign Application Priority Data
May 19, 2025 (CN) 202510646200.2

(51) Int. Cl. G16H 10/60

(52) U.S. Cl. CPC G16H 10/60

(57) ABSTRACT

One or more implementations of this specification provide electronic medical record generation methods, task execution methods, devices, media, and products. An example electronic medical record generation method includes: acquiring medical record content description text; evaluating, by using a trained task evaluation model, whether an electronic medical record generation task to be executed can be completed based on the medical record content description text, and further outputting to-be-supplemented information when a negative conclusion is obtained, where the to-be-supplemented information describes knowledge that needs to be supplemented to complete the electronic medical record generation task; obtaining through screening, from a predetermined medical knowledge base, supplementary knowledge indicated by the to-be-supplemented information; and inputting the medical record content description text and the supplementary knowledge to a trained electronic medical record generation model, so that the electronic medical record generation model executes the electronic medical record generation task to generate an electronic medical record.

### CROSS-REFERENCE TO RELATED APPLICATIONS

This application claims priority to Chinese Patent Application No. 202510646200.2, filed on May 19, 2025, which is hereby incorporated by reference in its entirety.

### TECHNICAL FIELD

One or more implementations of this specification relate to the field of terminal technologies, and in particular, to electronic medical record generation methods, task execution methods, electronic devices, computer-readable media, and computer program products.

### BACKGROUND

With the continuous development of medical informatization, electronic medical records already become an indispensable tool in clinical practice. However, conventional medical record entry methods rely on manual input, which is not only time-consuming and labor-intensive but also error-prone, making it difficult to satisfy growing medical demands. In busy clinical environments, doctors often cannot spare sufficient time for detailed medical record documentation, resulting in uneven quality of medical records.

### SUMMARY

In view of this, one or more implementations of this specification provide electronic medical record generation methods, task execution methods, electronic devices, computer-readable media, and computer program products.

To achieve the above objectives, one or more implementations of this specification provide the following technical solutions.

According to a first aspect of one or more implementations of this specification, an electronic medical record generation method is provided, including: acquiring medical record content description text; evaluating, by using a trained task evaluation model, whether an electronic medical record generation task to be executed can be completed based on the medical record content description text, and further outputting to-be-supplemented information when a negative conclusion is obtained, where the to-be-supplemented information is used to describe knowledge that needs to be supplemented to complete the electronic medical record generation task; obtaining through screening, from a predetermined medical knowledge base, supplementary knowledge indicated by the to-be-supplemented information; and inputting the medical record content description text and the supplementary knowledge to a trained electronic medical record generation model, so that the electronic medical record generation model executes the electronic medical record generation task to generate an electronic medical record.

According to a second aspect of one or more implementations of this specification, a task execution method is provided, including: acquiring task description information; evaluating, by using a trained task evaluation model, whether a target task to be executed can be completed based on the task description information, and further outputting to-be-supplemented information when a negative conclusion is obtained, where the to-be-supplemented information is used to describe knowledge that needs to be supplemented to complete the target task; obtaining through screening, from a predetermined knowledge base, supplementary knowledge indicated by the to-be-supplemented information; and inputting the task description information and the supplementary knowledge to a trained task execution model, so that the task execution model executes the target task to obtain a task execution result.

According to a third aspect of one or more implementations of this specification, an electronic device is provided, including: a processor; and a storage, configured to store processor-executable instructions, where the processor, when executing the executable instructions, is configured to implement the method according to the first aspect or the second aspect.

According to a fourth aspect of one or more implementations of this specification, a computer-readable storage medium is provided, where the computer-readable storage medium stores a computer program, and the program, when executed by a processor, implements the steps of the method according to the first aspect or the second aspect.

According to a fifth aspect of one or more implementations of this specification, a computer program product is provided, including a computer program, where the computer program, when executed by a processor, implements the steps of the method according to the first aspect or the second aspect.

The technical solutions provided by the implementations of this specification may include the following beneficial effects.

In implementations of this specification, whether the medical record content description text satisfies a demand for electronic medical record generation is evaluated by using the task evaluation model, and further the medical knowledge can be intelligently supplemented from the predetermined medical knowledge base when the task evaluation model obtains the negative conclusion and the to-be-supplemented information. In addition, the to-be-supplemented information output by the task evaluation model has stronger pertinence and directionality, which can improve the quality of medical knowledge supplementation. Then, the medical record content description text and the supplementary knowledge are input to the trained electronic medical record generation model, to ensure that medical record content generated by a final electronic medical record generation model is accurate and complete, thereby improving medical record quality.

It should be understood that the above-mentioned general descriptions and the following detailed descriptions are merely examples and explanations, and are not intended to limit this specification.

### BRIEF DESCRIPTION OF DRAWINGS

FIG. 1 is a flowchart of an electronic medical record generation method according to an example implementation;

FIG. 2 is another flowchart of an electronic medical record generation method according to an example implementation;

FIG. 3 is a diagram of electronic medical record quality evaluation and display according to an example implementation;

FIG. 4 is a flowchart of a task execution method according to an example implementation; and

FIG. 5 is a diagram of a structure of an electronic device according to an example implementation.

### DESCRIPTION OF IMPLEMENTATIONS

Example implementations are described in detail here, and examples of the example implementations are presented in the accompanying drawings. When the following description relates to the accompanying drawings, unless specified otherwise, same numbers in different accompanying drawings represent same or similar elements. Implementations described in the following example implementations do not represent all implementations consistent with one or more implementations of this specification. On the contrary, the implementations are merely examples of apparatuses and methods that are described in the appended claims in detail and consistent with some aspects of one or more implementations of this specification.

It is worthwhile to note that, in some other implementations, the steps of the corresponding method are not necessarily performed in the order shown and described in this specification. In some other implementations, the method can include more or less steps than those described in this specification. In addition, a single step described in this specification may be divided into a plurality of steps for description in other implementations, and a plurality of steps described in this specification may also be combined into a single step for description in other implementations.

User information (including but not limited to user equipment information, personal information of a user, etc.) and data (including but not limited to data used for analysis, stored data, displayed data, etc.) used in this specification are information and data that are authorized by the user or fully authorized by each party, related data needs to be collected, used, and processed by abiding by related laws and regulations and standards of a related country and region, and a corresponding operation entry is provided, so that the user chooses to perform authorization or rejection.

Relevant terms mentioned in implementations of this specification are explained here.
1. A language model, such as a large language model (LLM) or a BERT model, refers to an artificial intelligence model based on a deep learning technology, especially trained by using a large corpus, aimed at understanding and generating text similar to a human language, with powerful natural language understanding and generation capabilities. A target of the language model is to achieve various applications through natural language processing capabilities, such as text generation, translation, summarization, question answering, and dialogue systems, thereby helping improve the efficiency of human-computer interaction and a degree of automation.
2. A prompt refers to a type of prompt, guidance, or indication. In the field of artificial intelligence (AI), a role of the prompt is to guide and direct the language model to generate a specific type of output. The output can be a text fragment or question used to inspire the language model to think and generate relevant content.

Based on problems in related technologies, the implementations of this specification provide electronic medical record generation methods to realize automatic generation of electronic medical records.

Models used in the implementations of this specification, such as a task evaluation model, an electronic medical record generation model, a quality evaluation model, and a speech recognition model, are described by using examples below.
(1) The task evaluation model is used to evaluate whether an electronic medical record generation task to be executed can be completed based on the medical record content description text, and further output to-be-supplemented information when a negative conclusion is obtained, where the to-be-supplemented information is used to describe knowledge that needs to be supplemented to complete the electronic medical record generation task.

In a possible implementation, a pre-trained language model in a related technology can be directly used for the task evaluation model.

In another possible implementation, to improve evaluation accuracy, the task evaluation model can be obtained by fine-tuning the pre-trained language model based on a first training dataset. The first training dataset includes a positive sample and a negative sample. The positive sample includes medical record content description text used as a model input (the text includes complete key information in content), and a supervised label of the positive sample includes a positive conclusion (for example, “Information is complete, the electronic medical record generation task can be directly executed”). The negative sample includes medical record content description text used as a model input (the text lacks some key information in content, for example, missing a medical history, a medication record, and a test result), and a supervised label of the negative sample includes a negative conclusion and the to-be-supplemented information (for example, “Patient past medical history information is missed, and this part of content needs to be supplemented”).

During a fine-tuning training process, an electronic device inputs the sample (the positive sample or the negative sample) included in the first training dataset to a task evaluation model to be trained. The task evaluation model generates a corresponding prediction output, specifically including a judgment on whether the input medical record content description text is sufficient to complete the electronic medical record generation task (namely, a predicted conclusion), and outputs information that needs to be supplemented (namely, predicted supplementary information) when the judgment is the negative conclusion. A prediction result output by the model is compared with a supervised label corresponding to the input sample.

An error between the prediction result and the supervised label is calculated to evaluate accuracy and standardization of an output of a current task evaluation model. Specifically, a combination of loss metrics suitable for multi-task output can be used, for example, a cross-entropy loss is used for a classification task and a sequence generation loss function (for example, a token-level cross-entropy loss or a KL divergence) is used for text generation or an extraction task. The error reflects a degree of deviation of the model in determining information completeness and generating supplementary content.

With a training target of minimizing the error between the prediction result and the supervised label, a trainable parameter in the task evaluation model is automatically adjusted through a backpropagation algorithm. Each parameter update is based on a principle of “minimizing the error”, thereby promoting the model to gradually learn which key information in the input text plays a decisive role in task completion and which specific content should be supplemented when missing.

The fine-tuning process is usually performed in a plurality of iterations. As a quantity of training epochs increases, an output of the model becomes closer to actual annotation, and a prediction error continuously decreases. Finally, the task evaluation model converges into a model that can accurately determine whether the medical record description text satisfies a demand of the electronic medical record generation task and provides a clear supplementary suggestion when necessary, thereby effectively improving accuracy and an intelligence level of an overall generation process.
(2) The electronic medical record generation model is used to execute the electronic medical record generation task based on input content to generate an electronic medical record.

In a possible implementation, a pre-trained language model in a related technology can be directly used for the electronic medical record generation model.

In another possible implementation, to improve accuracy, the electronic medical record generation model can be obtained by fine-tuning the pre-trained language model based on a second training dataset, where the second training dataset includes text content used as a model input (such as a medical record, a doctor's note, a test result, and a chief complaint of a patient), and an electronic medical record used as a supervised label.

Similar to the above-mentioned fine-tuning process, the electronic device inputs each piece of text content used as a model input from the second training dataset to an electronic medical record generation model to be trained, and the model generates a predicted electronic medical record based on the input text content. The predicted electronic medical record is compared with a supervised label corresponding to the input text content (namely, real electronic medical record text), and an error between the two is calculated to evaluate accuracy and standardization of a medical record generation capability of a current model. A training target is to minimize a difference between the predicted electronic medical record and the real electronic medical record. A trainable parameter in the model is automatically updated based on a current error through a backpropagation algorithm. Each parameter update follows a principle of minimizing the error, which helps the model gradually learn how to effectively convert structured and semi-structured input information to complete, compliant, and medically standardized electronic medical record text.

The entire fine-tuning process is performed by using an iterative optimization method, usually repeating a training cycle of input-prediction-evaluation-update in a plurality of training rounds. As training progresses, the electronic medical record text generated by the model becomes closer to a real record, and an error gradually decreases until converge. A finally obtained electronic medical record generation model can accurately understand and extract key medical information from the input content, and generate electronic medical record text with a fluent language, complete content, and a reasonable structure.
(3) The quality evaluation model is used to evaluate quality of the electronic medical record in at least one quality dimension, to obtain quality evaluation information of the electronic medical record in all quality dimensions. The quality dimensions include but are not limited to at least one of the following: accuracy, completeness, professionalism, and readability.

In a possible implementation, a pre-trained language model in a related technology can be directly used for the quality evaluation model.

In another possible implementation, to improve accuracy, the quality evaluation model is obtained by fine-tuning the pre-trained language model based on a third training dataset, where the third training dataset includes an electronic medical record used as a model input, and quality evaluation information of the electronic medical record used as a supervised label in at least one quality dimension. For example, a form of discrete labels (for example, excellent/good/fair/poor) or continuous scores (for example, a score in a range of 0.0 to 1.0) can be used for the quality evaluation information. Implementations are not limited in this implementation.

Similar to the above-mentioned fine-tuning process, the electronic device inputs each electronic medical record used as a model input in the third training dataset to a quality evaluation model to be trained, and the model scores or classifies the performance of the input electronic medical record in all quality dimensions based on the input electronic medical record, to generate a corresponding prediction evaluation. The electronic device then compares the prediction evaluation with a corresponding supervised label (quality evaluation information of the electronic medical record in at least one quality dimension) and calculates an error, where the error is used as a loss function input in the training process, and the electronic device automatically adjusts all trainable parameters in the model through a backpropagation algorithm. Each parameter update aims to “minimize the error between the prediction evaluation and the supervised label”, thereby prompting the model to gradually learn determining standards for different quality dimensions and corresponding text features.

In the training process, the model continuously iterates and performs a training cycle of “prediction-comparison-optimization”. As a quantity of training epochs increases, a quality evaluation capability of the model for the electronic medical record in various dimensions continuously improves, and the error gradually decreases until the model converges.
(4) The speech recognition model is used to recognize a speech signal related to generating the electronic medical record, to obtain medical record content description information.

In a possible implementation, a trained speech recognition model in a related technology can be directly used for the speech recognition model.

In another possible implementation, to improve recognition accuracy for a speech signal in a medical scenario, the speech recognition model is obtained through training based on a fourth training dataset, where the fourth training dataset includes a speech signal sample that is related to the medical scenario and that is used as a model input (for example, a doctor's ward round recording, a dictated medical history, and a consultation record), and speech recognition text used as a supervised label. The electronic device inputs each speech signal sample in the fourth training dataset to a speech recognition model to be trained, where the model can map a voice feature to a corresponding text sequence and output predicted text; the electronic device compares the predicted text output by the model with a corresponding supervised label (real speech recognition text) and calculates an error, where the error is used as a loss function input in the training process; and the electronic device automatically adjusts all trainable parameters in the model through a backpropagation algorithm. A training target is to minimize the error between the predicted text output by the model and the corresponding supervised label, to improve accuracy of voice-to-text conversion. During the training, a model parameter is automatically updated based on a current loss by using the backpropagation algorithm. A training process is usually performed in a plurality of iterations. In each round, the model performs a cycle of “prediction-evaluation-update” on a new batch of samples, to continuously optimize a speech recognition capability of the model.

Since training samples include the speech signal sample related to the medical scenario, the adaptability of the speech recognition model to medical context is enhanced, thereby improving a capability of recognizing proper nouns in the medical scenario by the model. After the training is completed, an obtained speech recognition model can automatically and accurately recognize a voice input in the medical scenario as the medical record content description information, to provide a reliable text input foundation for a subsequent electronic medical record generation task.

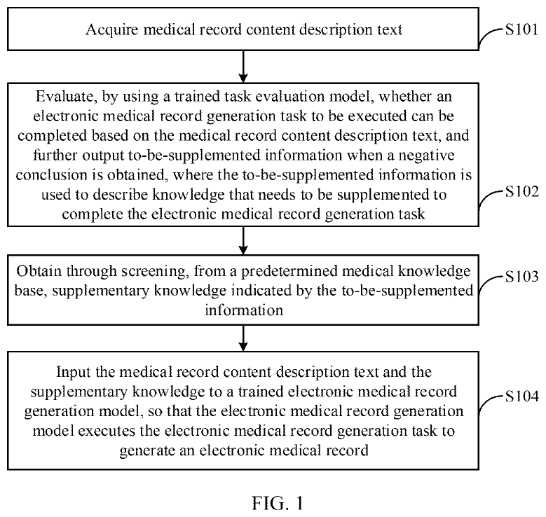

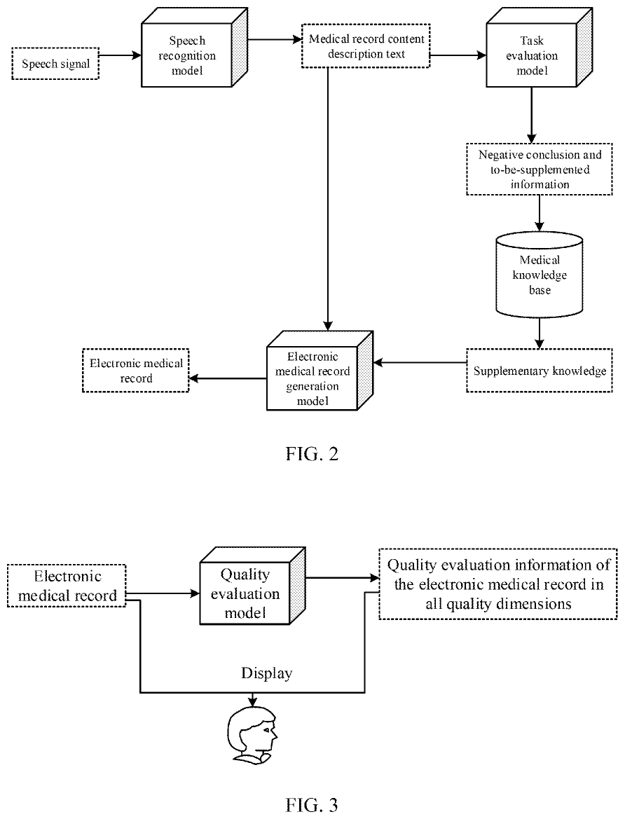

Referring to FIG. 1 and FIG. 2, an electronic medical record generation method according to this specification is described by using examples below. The electronic medical record generation method can be performed by an electronic device. The electronic device includes but is not limited to a smart phone/mobile phone, a tablet computer, a personal digital assistant (PDA), a laptop computer, a desktop computer, a physical server, a virtual server, or a cloud server, etc. Implementations are not limited in this implementation. The electronic medical record generation method includes the following:
S101: Acquire medical record content description text.

In a possible implementation, referring to FIG. 2, the electronic device can receive a speech signal related to generating an electronic medical record, and then perform speech recognition on the speech signal by using a trained speech recognition model to obtain the medical record content description text.

In another possible implementation, the electronic device also supports a doctor in directly entering the medical record content description text into the electronic device. An input method can be diversified, and an input style is not strictly limited in this implementation, and can be specifically selected based on an actual application scenario.

For example, the medical record content description text can include but is not limited to at least one of the following: symptom description, disease summary, basic patient information, a test and examination result, a preliminary diagnosis and treatment opinion, a task type of the electronic medical record generation task, and a format demand of the electronic medical record.
S102: Evaluate, by using a trained task evaluation model, whether the electronic medical record generation task to be executed can be completed based on the medical record content description text, and further output to-be-supplemented information when a negative conclusion is obtained, where the to-be-supplemented information is used to describe knowledge that needs to be supplemented to complete the electronic medical record generation task.

In this step, at least the “medical record content description text” obtained in S101 is input to the task evaluation model. The task evaluation model first performs a completeness evaluation on the medical record content description text, in other words, whether the medical record content description text includes key information needed to generate whether electronic medical record. If the evaluation is “The information is complete”, the task evaluation model directly outputs a positive conclusion. If the evaluation is “The information is incomplete”, the task evaluation model further generates the to-be-supplemented information, and finally outputs a negative conclusion and the to-be-supplemented information.

Given that the medical record content description text may have problems such as rough expression, incomplete information, or non-standard expression, the implementations of this specification introduce the task evaluation model to pre-evaluate the medical record content description text, to determine whether the medical record content description text has sufficient information to be used to execute the electronic medical record generation task. Compared with a method of directly generating the electronic medical record based on original text, in this solution, an information gap in the text can be effectively identified, thereby avoiding an inaccurate medical record generation result caused by incomplete input content, and improving overall generation quality and applicability.

For example, it is considered that the electronic medical record generation task also has a plurality of task types, including but not limited to an outpatient medical record, an admission summary, an inpatient medical record, a discharge summary, a surgical record, and an informed consent form. To improve evaluation accuracy, the electronic device can first perform intent recognition on the medical record content description text to determine a task type of the electronic medical record generation task, and then input the medical record content description text and the task type to the task evaluation model, so that the task evaluation model evaluates whether the electronic medical record generation task indicated by the task type can be completed based on the medical record content description text.

In this implementation, introducing an intent recognition mechanism to determine a specific task type of the electronic medical record generation task can significantly improve the evaluation pertinence and accuracy of the task evaluation model. Specifically, different types of electronic medical records (such as an outpatient medical record, a discharge summary, and a surgical record) have significant differences in a content structure, an information focus, and an expression norm. If the task types are not distinguished in an evaluation process, it may lead to inconsistent evaluation standards, affecting the reliability of evaluation results. The task type is first identified and input as an auxiliary input together with the medical record content description text to the task evaluation model, so that the model can perform accurate determining based on a standard demand of a corresponding type, and identify whether current input content satisfies a condition for generating this type of electronic medical record. This method not only enhances a capability of understanding the evaluation context by the model but also effectively avoids misjudgments caused by an ambiguous task type, thereby improving the evaluation adaptability and output quality of an overall system in a multi-task electronic medical record generation scenario.

In one case, it is considered that the task evaluation model already learns and masters content demands and determining standards for electronic medical record generation tasks corresponding to different task types after the above-mentioned fine-tuning process. Therefore, in actual applications, the medical record content description text and the corresponding task type can be directly provided as an input to the task evaluation model. With the help of the model's internalized knowledge about the task types, the model can perform a targeted evaluation on input text in conjunction with a task type, thereby accurately determining whether the input text satisfies a condition for completing this type of electronic medical record generation task, without relying on an additional rule or template matching process, and thereby simplifying a system process and improving evaluation efficiency.

In another case, to further improve evaluation accuracy, a plurality of prompt templates can be pre-configured, where each of the prompt templates corresponds to one task type and describes an input demand of an electronic medical record generation task indicated by the task type. The electronic device can select a target prompt template matching the task type from the pre-configured plurality of prompt templates based on the task type, then embed the medical record content description text and the task type into the target prompt template to obtain a target prompt, and finally input the target prompt to the task evaluation model.

Introducing a prompt template mechanism corresponding to the task type can further enhance understanding and determining capabilities of the task evaluation model for different electronic medical record generation tasks. For example, each of the prompt templates can describe content related to a specific task type, providing clear and structured task background information for the task evaluation model. Embedding the medical record content description text and the task type into the matching prompt template to generate the target prompt and using the target prompt as an input to be transferred to the task evaluation model can effectively guide the task evaluation model to focus on a key content element and a determining standard that are needed for the specific task type. This method not only improves a capability of perceiving input context by the task evaluation model but also enhances controllability and consistency of the evaluation process, thereby still maintaining evaluation accuracy and robustness when dealing with complex or ambiguously expressed medical record content, and helping perform more reliable pre-validation for electronic medical record generation.

A discharge summary is used as an example for illustration, and a prompt template corresponding to the discharge summary can be predetermined as shown in the following table:

Please determine, based on the following medical record content, whether the medical
record content already includes key information needed to complete the discharge summary,
including but not limited to content such as a cause for admission, a main diagnosis and
treatment process, a condition upon discharge, and a medical advice and follow-up visit
recommendation. If the information is incomplete, please indicate specific content that needs
to be supplemented.
[Medical record content]: {Medical record content description text}
[Task type]: Discharge summary

The prompt template clearly describes input demands for an electronic medical record generation task corresponding to the “discharge summary” (for example, a cause for admission, a diagnosis and treatment process, a condition upon discharge, and a medical advice and recommendation), thereby guiding the task evaluation model to determine completeness and applicability of the input text around these key points.
S103: Obtain through screening, from a predetermined medical knowledge base, supplementary knowledge indicated by the to-be-supplemented information.

For example, the predetermined medical knowledge base can include but is not limited to at least one of the following:
(1) At least one of basic medical knowledge, a professional term explanation, and an indicator interpretation for various departments, for example, including general terms for various professions of internal medicine, surgery, obstetrics and gynecology, pediatrics, neurology, oncology, etc., definitions of common diseases, normal ranges and abnormal interpretations of test indicators (such as a blood routine, a liver function, and an imaging result).
(2) Specific demands of different electronic medical record templates, including but not limited to, a field structure, a content specification, and a compliance demand for document templates such as an outpatient medical record, an inpatient medical record, a discharge summary, a surgical record, and an informed consent form. For example, the discharge summary should include a diagnostic basis and treatment plan summary; and the surgical record should include an intraoperative finding and a postoperative condition.
(3) Latest knowledge in various medical specialties and standard medical professional knowledge, such as a clinical pathway, a diagnosis and treatment guideline (for example, “Guidelines for the Prevention and Treatment of Hypertension”), a drug usage specification, and a medical journal abstract.
(4) Case examples and expert-annotated samples, including typical case document samples reviewed by experts, helping the medical record generation model refer to a real expression method.
(5) Common patient questions and answers and health education content, including content of daily explanations by doctors, such as “Is fasting needed before surgery?” and “When is the best time to take blood pressure medication?”, enhancing reasonableness of a patient communication part or a health education advice part in the medical record.
(6) A medical entity dictionary and a relationship graph, including standard names and relationship networks of entities such as a disease, a symptom, a drug, a test, and a treatment, facilitating improvement of professionalism and consistency of the model.

For example, the predetermined medical knowledge base can be represented in a form of at least one of a frequently asked questions (FAQ) knowledge base, a knowledge graph, a search engine, and a structured database, but is not limited thereto. The FAQ knowledge base includes a structured question-answer pair set, facilitating quick retrieval of common knowledge. The knowledge graph constructs a medical knowledge network in a form of entity-attribute-relationship, supporting complex reasoning and semantic retrieval. The structured database stores medical standard entries (such as a drug information table) by using a relational database, facilitating data table-driven information retrieval. The search engine supports keyword search or semantic search, facilitating quick search of a document or a paragraph.

In this step, the obtaining of the supplementary knowledge through screening is based on the “to-be-supplemented information” output by the task evaluation model in S102, rather than being directly obtained through screening based on the medical record content description text. Compared with original medical record content description text, the to-be-supplemented information output by the task evaluation model has stronger pertinence and directionality, can clearly indicate specific knowledge points or a content type missed from a current electronic medical record generation task, thereby providing a more focused query condition for screening. This method effectively avoids a screening error caused by redundant information or semantically ambiguity in medical record descriptions, and significantly improves accuracy and relevance of extracting the supplementary knowledge from the predetermined medical knowledge base.

In a possible implementation, the electronic device can acquire a first embedding vector converted from the to-be-supplemented information, and acquire second embedding vectors converted from respective pieces of knowledge in the medical knowledge base; and then obtain, based on a similarity between the first embedding vector and each of the second embedding vectors, the supplementary knowledge indicated by the to-be-supplemented information. The electronic device can achieve a more efficient and semantically accurate supplementary knowledge screening process by respectively converting the to-be-supplemented information and knowledge entries in the medical knowledge base into embedding vectors and performing matching based on a similarity in embedding space. Vectorized representation can capture a semantic-level similarity, and even if there is a textual difference between the to-be-supplemented information and the knowledge entries, a potential semantic association can still be identified, thereby improving accuracy and intelligence of screening.
S104: Input the medical record content description text and the supplementary knowledge to a trained electronic medical record generation model, so that the electronic medical record generation model executes the electronic medical record generation task to generate the electronic medical record.

In a possible implementation, it is considered that the electronic medical record generation model already learns and masters an electronic medical record generation capability after the above fine-tuning process. Therefore, in actual applications, the medical record content description text and the supplementary knowledge can be directly provided as an input to the electronic medical record generation model. With the help of the model's internalized knowledge about the task types, the model can perform a targeted evaluation on input text in conjunction with a task type, thereby accurately determining whether the input text satisfies a condition for completing this type of electronic medical record generation task, without relying on an additional rule or template matching processes, and thereby simplifying a process and improving evaluation efficiency.

In another case, to further improve evaluation accuracy, a plurality of prompt templates can be pre-configured, where each of the prompt templates corresponds to one task type and describes at least one execution step of an electronic medical record generation task indicated by the task type. The electronic device can select a target prompt template matching the task type from the pre-configured plurality of prompt templates based on the task type, then embed the medical record content description text, the task type, and a supplementary indication to the target prompt template to obtain a target prompt, and finally input the target prompt to the electronic medical record generation model, so that the electronic medical record generation model executes the electronic medical record generation task to generate the electronic medical record. In this implementation, different types of electronic medical records (such as an outpatient medical record and a discharge summary) have different information organization structures and content focuses. The prompt template is used to clarify the execution step for the task type, to guide the model to focus on a specific element during generation, thereby improving relevance and pertinence of output content.

According to the electronic medical record generation method in this implementation, automatic generation of the electronic medical record is implemented, whether the medical record content description text satisfies a demand for electronic medical record generation is evaluated by the task evaluation model, the medical knowledge can be intelligently supplemented from the predetermined medical knowledge base when the task evaluation model obtains the negative conclusion and outputs the to-be-supplemented information, and the medical record content description text and the supplementary knowledge are input to the trained electronic medical record generation model, to ensure that the medical record content generated by a final electronic medical record generation model is accurate and complete, thereby improving medical record quality.

In some implementations, when the task evaluation model outputs the positive conclusion, the medical record content description text can be directly input to the trained electronic medical record generation model, so that the electronic medical record generation model executes the electronic medical record generation task to generate the electronic medical record.

In some implementations, referring to FIG. 3, after the electronic medical record generation model outputs the electronic medical record, the electronic device can input the electronic medical record to a trained quality evaluation model, so that the quality evaluation model evaluates quality of the electronic medical record in at least one quality dimension and outputs quality evaluation information of the electronic medical record in all quality dimensions. The quality dimensions include but are not limited to at least one of the following: accuracy, completeness, professionalism, and readability.

In a possible implementation, the electronic device can automatically evaluate whether the quality evaluation information of the electronic medical record in all the quality dimensions satisfies a predetermined quality demand, and if quality evaluation information of at least one quality dimension does not satisfy the predetermined quality demand, the electronic medical record generation task can be re-executed based on the quality evaluation information that does not satisfy the predetermined quality demand and the medical record content description text. Specifically, re-execution methods can include the following two types: One method is to perform regeneration starting from the beginning, for example, re-entering the task evaluation process: inputting the medical record content description text to the task evaluation model again, determining whether the supplementary knowledge is needed, performing update with the input accordingly, and then regenerating the electronic medical record; and the other method is to perform modification based on a current electronic medical record: using quality evaluation information that does not satisfy the predetermined quality demand as guidance information, inputting the quality evaluation information together with the electronic medical record and the medical record content description text to the electronic medical record generation model, indicating the electronic medical record generation model to perform targeted optimization and modification on only a part of the electronic medical record that has a quality problem. The predetermined quality demand can be specifically set based on an actual application scenario. Implementations are not limited in this implementation.

In this implementation, an automatic error correction and enhanced medical record quality control mechanism is implemented, and a quality defect can be discovered and repaired without manual intervention, so that closed-loop verification and optimization of a multi-dimensional quality indicator are realized, and that an output document satisfies both clinical and document specification demands is ensured, thereby improving an autonomous intelligence level of a system and reducing a risk of a low-quality output.

In another possible implementation, referring to FIG. 3, at least one of the electronic medical record or the quality evaluation information of the electronic medical record in all the quality dimensions can be displayed to a user (such as a doctor). If dissatisfied with a certain part of generated content (such as a treatment plan description and a test result record), the user can indicate the electronic device to perform modification, for example, by using a voice description method, but not limited thereto, or the user can directly perform modification manually. If receiving modification indication information for at least one piece of medical record content in the electronic medical record, the electronic device can re-execute the electronic medical record generation task based on the modification indication information and the medical record content description text. Specifically, re-execution methods can include the following two types: One method is to perform regeneration starting from the beginning, for example, re-entering the task evaluation process: inputting the medical record content description text to the task evaluation model again, determining whether the supplementary knowledge is needed, performing update with the input accordingly, and then regenerating the electronic medical record; and the other method is to perform modification based on a current electronic medical record: using the modification indication information as guidance information, inputting the quality evaluation information together with the electronic medical record and the medical record content description text to the electronic medical record generation model, indicating the electronic medical record generation model to perform targeted optimization and modification on only a part of the electronic medical record that has a quality problem. This implementation retains space for manual review and customized modification, enhances a capability of responding to a subjective intention of a doctor, combines an advantage of human-computer collaboration, and improves personalization and actual application value of content of the electronic medical record.

Various technical features in the above-mentioned implementations can be randomly combined, provided that there is no conflict or contradiction between combinations of the features. The combinations are not described one by one due to a space limitation. Therefore, any combination of the various technical features in the above-mentioned implementations also falls within the scope disclosed in this specification.

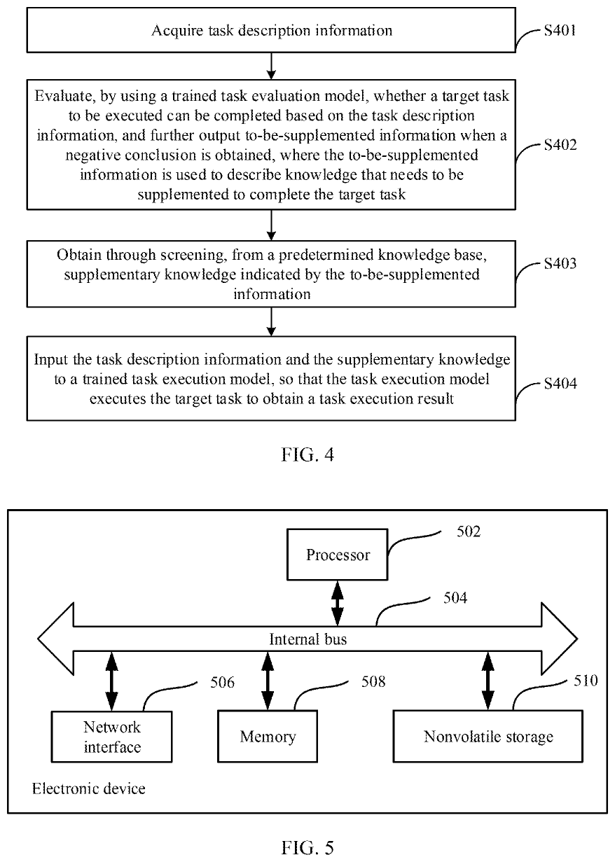

In some implementations, it is considered that implementation logic of the above-mentioned electronic medical record generation task has strong generality, and is not only applicable to the electronic medical record generation task but can also be extended to more types of task execution scenarios. For example, in various applications that need generating complex text or performing decision-making reasoning based on input information (such as legal document drafting, intelligent customer service question answering, and academic abstract generation), a collaboration mechanism of task evaluation, knowledge supplementation, and model execution can also improve completeness and accuracy of task execution. Therefore, referring to FIG. 4, an implementation of this specification further provides a task execution method, including the following:
S401: Acquire task description information.
S402: Evaluate, by using a trained task evaluation model, whether a target task to be executed can be completed based on the task description information, and further output to-be-supplemented information when a negative conclusion is obtained, where the to-be-supplemented information is used to describe knowledge that needs to be supplemented to complete the target task.
S403: Obtain through screening, from a predetermined knowledge base, supplementary knowledge indicated by the to-be-supplemented information.
S404: Input the task description information and the supplementary knowledge to a trained task execution model, so that the task execution model executes the target task to obtain a task execution result.

For example, the task description information includes report content description information, the target task includes a report generation task, and the task execution result includes a report.

For example, in a civil dispute case, a lawyer expects to quickly generate a compliant document that conforms to a specification. The electronic device executes the following steps according to the method shown in FIG. 4.
S401: Acquire task description information: “On March 2023, Party Zhang borrowed RMB 200,000 from Party Li, with a repayment deadline of Sep. 30, 2023, but Li has not repaid the loan to date.”
S402: Use the task evaluation model for evaluation: The task evaluation model determines that the description does not include complete complaint elements, cannot directly generate a complaint, and therefore outputs to-be-supplemented information, for example, “Is there a written agreement or evidence for the loan?”, “Are there any collection record?”, “Is basic identification information of the parties clear?”, and “Is interest requested to be adjudicated by the court?”.
S403: Supplement knowledge: Based on the to-be-supplemented information, relevant content is obtained through screening from a legal knowledge base, for example, a standard format and a content demand for the complaint; an evidentiary element for a private lending case; an example of a party identification description in the complaint; a legal basis for claiming interest.
S404: Generate a report (the complaint): The task description information and the supplementary knowledge are input to the task execution model (for example, a legal text generation model fine-tuned from a large language model) to generate a civil complaint that is well-structured, logically clear, and compliant with a court demand, including: plaintiff and defendant information; a fact and a reason; a claim; a list of evidence; a legal basis.

In this method, a case in which an invalid complaint is generated due to incomplete information is avoided, to-be-supplemented content can be automatically indicated by a prompt and evidence supplementation can be guided, thereby significantly improving generation quality and work efficiency of a legal document, and lowering a professional legal threshold, particularly suitable for scenarios such as a legal aid platform and an intelligent legal consultation system.

In an implementation, the method further includes: performing intent recognition on the task description information to determine a task type of the target task. Evaluating, by using the trained task evaluation model, whether the target task to be executed can be completed based on the task description information includes: inputting the task description information and the task type to the task evaluation model, so that the task evaluation model evaluates whether the target task indicated by the task type can be completed based on the task description information. In an implementation, the method further includes: selecting, based on the task type, a target prompt template matching the task type from a plurality of predetermined prompt templates, where each of the prompt templates corresponds to one task type and describes an input demand of the target task indicated by the task type; and embedding the task description information and the task type into the target prompt template to obtain a target prompt. Inputting the task description information and the task type to the task evaluation model includes: inputting the target prompt to the task evaluation model.

In an implementation, obtaining through screening, from the predetermined knowledge base, the supplementary knowledge indicated by the to-be-supplemented information includes: acquiring a first embedding vector converted from the to-be-supplemented information, and acquiring second embedding vectors converted from respective pieces of knowledge in the knowledge base; and obtaining, based on a similarity between the first embedding vector and each of the second embedding vectors, the supplementary knowledge indicated by the to-be-supplemented information.

In an implementation, the method further includes: inputting the task execution result to a trained quality evaluation model, so that the quality evaluation model evaluates quality of the task execution result in at least one quality dimension and outputs quality evaluation information of the task execution result in all quality dimensions.

In an implementation, the method further includes: re-executing, if quality evaluation information of at least one quality dimension does not satisfy a predetermined quality demand, the target task based on the quality evaluation information that does not satisfy the predetermined quality demand and the task description information.

In an implementation, the method further includes: displaying at least one of the task execution result or the quality evaluation information of the task execution result in all the quality dimensions; and re-executing, if modification indication information for at least one piece of content in the task execution result is received, the target task based on the modification indication information and the task description information.

In an implementation, the task evaluation model is obtained by fine-tuning a pre-trained language model based on a first training dataset, where the first training dataset includes a positive sample and a negative sample; the positive sample includes task description information used as a model input, and a supervised label of the positive sample includes a positive conclusion; and the negative sample includes task description information used as a model input, and a supervised label of the negative sample includes a negative conclusion and the to-be-supplemented information.

In an implementation, the task execution model is obtained by fine-tuning a pre-trained language model based on a second training dataset, where the second training dataset includes text content used as a model input and a task execution result used as a supervised label.

In an implementation, the quality evaluation model is obtained by fine-tuning a pre-trained language model based on a third training dataset, where the third training dataset includes a task execution result used as a model input, and quality evaluation information of the task execution result used as a supervised label in at least one quality dimension.

In an implementation, acquiring the task description information includes: receiving a speech signal related to the target task; and performing speech recognition on the speech signal by using a trained speech recognition model to obtain the task description information, where the speech recognition model is obtained through training based on a fourth training dataset, and the fourth training dataset includes a speech signal sample that is related to a scenario of the target task and that is used as a model input, and speech recognition text used as a supervised label.

In some implementations, an implementation of this specification further provides an electronic device, including: a processor; and a storage configured to store processor-executable instructions, where the processor runs the executable instructions to implement any one of the above-mentioned methods.

FIG. 5 is a diagram illustrating a structure of a device, according to an example implementation. As shown in FIG. 5, in terms of hardware, the device includes a processor 502, an internal bus 504, a network interface 506, a memory 508, and a nonvolatile storage 510, and certainly may further include hardware needed for another function. One or more implementations of this specification can be implemented by using a software-based method. For example, the processor 502 reads a corresponding computer program from the nonvolatile storage 510 into the memory 508, and then runs the computer program. Certainly, in addition to a software implementation, one or more implementations of this specification do not exclude another implementation, for example, a logic device or a combination of hardware and software. That is, an execution body of the following processing procedure is not limited to each logical unit, and can be hardware or a logic device.

In some implementations, an electronic medical record generation apparatus can be applied to the device shown in FIG. 5 to implement the technical solutions of this specification. The electronic medical record generation apparatus can include: a description text acquisition module, configured to acquire medical record content description text; a task evaluation module, configured to evaluate, by using a trained task evaluation model, whether an electronic medical record generation task to be executed can be completed based on the medical record content description text, and further output to-be-supplemented information when a negative conclusion is obtained, where the to-be-supplemented information is used to describe knowledge that needs to be supplemented to complete the electronic medical record generation task; a supplementation module, configured to obtain through screening, from a predetermined medical knowledge base, supplementary knowledge indicated by the to-be-supplemented information; and an electronic medical record generation module, configured to input the medical record content description text and the supplementary knowledge to a trained electronic medical record generation model, so that the electronic medical record generation model executes the electronic medical record generation task to generate an electronic medical record.

In some implementations, a task execution apparatus can be applied to the device shown in FIG. 5 to implement the technical solutions of this specification. The task execution apparatus can include: a description information acquisition module, configured to acquire task description information; a task evaluation module, configured to evaluate, by using a trained task evaluation model, whether a target task to be executed can be completed based on the task description information, and further output to-be-supplemented information when a negative conclusion is obtained, where the to-be-supplemented information is used to describe knowledge that needs to be supplemented to complete the target task; a supplementation module, configured to obtain through screening, from a predetermined knowledge base, supplementary knowledge indicated by the to-be-supplemented information; and a task execution module, configured to input the task description information and the supplementary knowledge to a trained task execution model, so that the task execution model executes the target task to obtain a task execution result.

An implementation process of the functions and effects of each module in the above-mentioned apparatus is detailed in the implementation process of corresponding steps in the above-mentioned methods, and is not repeated here.

Based on the same concept as the above-mentioned method, this specification further provides an electronic device, including: a processor; and a storage, configured to store processor-executable instructions. The processor runs the executable instructions to implement the steps of the method in any one of the above-mentioned implementations.

Based on the same concept as the above-mentioned method, this specification further provides a computer-readable storage medium. The computer-readable storage medium stores computer instructions, and when the instructions are executed by a processor, the steps of the method in any one of the above-mentioned implementations are implemented.

The computer-readable medium includes persistent, non-persistent, removable, and non-removable media that can store information by using any method or technology. The information can be computer-readable instructions, a data structure, a program module, or other data. Examples of the computer storage medium include but are not limited to a phase change random access memory (PRAM), a static random access memory (SRAM), a dynamic random access memory (DRAM), another type of random access memory (RAM), a read-only memory (ROM), an electrically erasable programmable read-only memory (EEPROM), a flash memory or another memory technology, a compact disc read-only memory (CD-ROM), a digital versatile disc (DVD) or another optical storage, a cassette magnetic tape, a magnetic disk storage, a quantum memory, a graphene-based storage medium, another magnetic storage device, or any other non-transmission medium. The computer storage medium can be configured to store information that can be accessed by a computing device. Based on the definition in this specification, the computer-readable medium does not include transitory media (transitory media) such as a modulated data signal and carrier.

Based on the same concept as the above-mentioned method, this specification further provides a computer program product, including a computer program/instructions. When the computer program/instructions is/are executed by a processor, the steps of the method according to any one of the above-mentioned implementations are implemented.

The above-mentioned descriptions are only example implementations of one or more implementations of this specification, but are not intended to limit the one or more implementations of this specification. Any modification, equivalent replacement, improvement, etc. made without departing from the spirit and principle of the one or more implementations of this specification shall fall within the protection scope of the one or more implementations of this specification.

What is claimed is:

1. A method, comprising:
acquiring, by an electronic device, medical record content description text;
evaluating, by using a trained task evaluation model, whether an electronic medical record generation task to be executed is able to be completed based on the medical record content description text;
in response to that the electronic medical record generation task to be executed is not able to be completed based on the medical record content description text, outputting to-be-supplemented information, wherein the to-be-supplemented information is used to describe knowledge that needs to be supplemented to complete the electronic medical record generation task;
obtaining through screening, from a medical knowledge base, supplementary knowledge indicated by the to-be-supplemented information; and
inputting the medical record content description text and the supplementary knowledge to a trained electronic medical record generation model, wherein the trained electronic medical record generation model executes the electronic medical record generation task to generate an electronic medical record.

2. The method according to claim 1, further comprising:
performing intent recognition on the medical record content description text to determine a task type of the electronic medical record generation task; and
wherein evaluating, by using the trained task evaluation model, whether the electronic medical record generation task to be executed is able to be completed based on the medical record content description text comprises:
inputting the medical record content description text and the task type to the trained task evaluation model, wherein the trained task evaluation model evaluates whether the electronic medical record generation task indicated by the task type is able to be completed based on the medical record content description text.

3. The method according to claim 2, further comprising:
selecting, based on the task type, a target prompt template matching the task type from a plurality of predetermined prompt templates, wherein each prompt template corresponds to one task type and describes an input demand of the electronic medical record generation task indicated by the task type; and
embedding the medical record content description text and the task type into the target prompt template to obtain a target prompt; and
wherein inputting the medical record content description text and the task type to the trained task evaluation model comprises:
inputting the target prompt to the trained task evaluation model.

4. The method according to claim 1, wherein obtaining through screening, from the medical knowledge base, the supplementary knowledge indicated by the to-be-supplemented information comprises:
acquiring a first embedding vector converted from the to-be-supplemented information, and acquiring second embedding vectors converted from respective pieces of knowledge in the medical knowledge base; and
obtaining, based on a similarity between the first embedding vector and each of the second embedding vectors, the supplementary knowledge indicated by the to-be-supplemented information.

5. The method according to claim 1, further comprising:
inputting the electronic medical record to a trained quality evaluation model, wherein the trained quality evaluation model evaluates quality of the electronic medical record in at least one quality dimension and outputs quality evaluation information of the electronic medical record in all quality dimensions.

6. The method according to claim 5, further comprising:
re-executing, in response to that quality evaluation information of the at least one quality dimension does not satisfy a predetermined quality demand, the electronic medical record generation task based on the quality evaluation information that does not satisfy the predetermined quality demand and the medical record content description text.

7. The method according to claim 5, further comprising:
displaying at least one of the electronic medical record or the quality evaluation information of the electronic medical record in all quality dimensions; and
re-executing, in response to that modification indication information for at least one piece of medical record content in the electronic medical record is received, the electronic medical record generation task based on the modification indication information and the medical record content description text.

8. The method according to claim 1, wherein the trained task evaluation model is obtained by fine-tuning a pre-trained language model based on at least one of a first training dataset or a second training dataset,
wherein the first training dataset comprises a positive sample and a negative sample; the positive sample comprises first medical record content description text used as a first model input, and a first supervised label of the positive sample comprises a positive conclusion; and the negative sample comprises second medical record content description text used as a second model input, and a second supervised label of the negative sample comprises a negative conclusion and second to-be-supplemented information; and
wherein the second training dataset comprises text content used as a model input and an electronic medical record used as a supervised label.

9. The method according to claim 5, wherein the trained quality evaluation model is obtained by fine-tuning a pre-trained language model based on a third training dataset, wherein the third training dataset comprises an electronic medical record used as a model input, and quality evaluation information of the electronic medical record used as a supervised label in one or more quality dimensions.

10. The method according to claim 1, wherein acquiring the medical record content description text comprises:
receiving a speech signal related to generating the electronic medical record; and
performing speech recognition on the speech signal by using a trained speech recognition model to obtain the medical record content description text, wherein:
the trained speech recognition model is obtained through training based on a fourth training dataset, and the fourth training dataset comprises a speech signal sample that is related to a medical scenario and that is used as a model input, and speech recognition text that is used as a supervised label.

11. A method, comprising:
acquiring, by an electronic device, task description information;
evaluating, by using a trained task evaluation model, whether a target task to be executed is able to be completed based on the task description information;
in response to that the target task to be executed is not able to be completed based on the task description information, further outputting to-be-supplemented information, wherein the to-be-supplemented information is used to describe knowledge that needs to be supplemented to complete the target task;
obtaining through screening, from a predetermined knowledge base, supplementary knowledge indicated by the to-be-supplemented information; and
inputting the task description information and the supplementary knowledge to a trained task execution model, wherein the trained task execution model executes the target task to obtain a task execution result.

12. The method according to claim 11, wherein the task description information comprises report content description information, the target task comprises a report generation task, and the task execution result comprises a report.

13. An electronic device, comprising:
at least one processor; and
at least one storage that stores processor-executable instructions executable by the at least one processor to implement operations comprising:
acquiring, by an electronic device, medical record content description text;
evaluating, by using a trained task evaluation model, whether an electronic medical record generation task to be executed is able to be completed based on the medical record content description text;
in response to that the electronic medical record generation task to be executed is not able to be completed based on the medical record content description text, outputting to-be-supplemented information, wherein the to-be-supplemented information is used to describe knowledge that needs to be supplemented to complete the electronic medical record generation task;
obtaining through screening, from a medical knowledge base, supplementary knowledge indicated by the to-be-supplemented information; and
inputting the medical record content description text and the supplementary knowledge to a trained electronic medical record generation model, wherein the trained electronic medical record generation model executes the electronic medical record generation task to generate an electronic medical record.

14. The electronic device according to claim 13, wherein the operations comprise:
performing intent recognition on the medical record content description text to determine a task type of the electronic medical record generation task; and
wherein evaluating, by using the trained task evaluation model, whether the electronic medical record generation task to be executed is able to be completed based on the medical record content description text comprises:
inputting the medical record content description text and the task type to the trained task evaluation model, wherein the trained task evaluation model evaluates whether the electronic medical record generation task indicated by the task type is able to be completed based on the medical record content description text.

15. The electronic device according to claim 14, wherein the operations comprise:
selecting, based on the task type, a target prompt template matching the task type from a plurality of predetermined prompt templates, wherein each prompt template corresponds to one task type and describes an input demand of the electronic medical record generation task indicated by the task type; and
embedding the medical record content description text and the task type into the target prompt template to obtain a target prompt; and
wherein inputting the medical record content description text and the task type to the trained task evaluation model comprises:
inputting the target prompt to the trained task evaluation model.

16. The electronic device according to claim 13, wherein obtaining through screening, from the medical knowledge base, the supplementary knowledge indicated by the to-be-supplemented information comprises:
acquiring a first embedding vector converted from the to-be-supplemented information, and acquiring second embedding vectors converted from respective pieces of knowledge in the medical knowledge base; and
obtaining, based on a similarity between the first embedding vector and each of the second embedding vectors, the supplementary knowledge indicated by the to-be-supplemented information.

17. The electronic device according to claim 13, wherein the operations comprise:
inputting the electronic medical record to a trained quality evaluation model, wherein the trained quality evaluation model evaluates quality of the electronic medical record in at least one quality dimension and outputs quality evaluation information of the electronic medical record in all quality dimensions.

18. The electronic device according to claim 17, wherein the operations comprise:
re-executing, in response to that quality evaluation information of the at least one quality dimension does not satisfy a predetermined quality demand, the electronic medical record generation task based on the quality evaluation information that does not satisfy the predetermined quality demand and the medical record content description text.

19. The electronic device according to claim 13, wherein the trained task evaluation model is obtained by fine-tuning a pre-trained language model based on at least one of a first training dataset or a second training dataset,
wherein the first training dataset comprises a positive sample and a negative sample; the positive sample comprises first medical record content description text used as a first model input, and a first supervised label of the positive sample comprises a positive conclusion; and the negative sample comprises second medical record content description text used as a second model input, and a second supervised label of the negative sample comprises a negative conclusion and second to-be-supplemented information; and
wherein the second training dataset comprises text content used as a model input and an electronic medical record used as a supervised label.

20. The electronic device according to claim 13, wherein acquiring the medical record content description text comprises:
receiving a speech signal related to generating the electronic medical record; and
performing speech recognition on the speech signal by using a trained speech recognition model to obtain the medical record content description text, wherein:
the trained speech recognition model is obtained through training based on a fourth training dataset, and the fourth training dataset comprises a speech signal sample that is related to a medical scenario and that is used as a model input, and speech recognition text that is used as a supervised label.
